(19) United States
(12) Patent Application Publication
(10) Pub. No.: US 2018/0047158 A1
(43) Pub. Date: Feb. 15, 2018

(54) CHEST RADIOGRAPH (CXR) IMAGE ANALYSIS

(71) Applicants: Ramot at Tel-Aviv University Ltd., Tel-Aviv (IL); Tel HaShomer Medical Research Infrastructure and Services Ltd., Ramat-Gan (IL)

(72) Inventors: Ofer GEVA, Haifa (IL); Hayit GRENSPAIN, Tel-Aviv (IL); Sivan LIEBERMAN, Moshav Beit-HaLevi (IL); Eli KONEN, Ramat-Gan (IL)

(21) Appl. No.: 15/552,278

(22) PCT Filed: Feb. 18, 2016
(86) PCT No.: PCT/IL2016/050195

§ 371 Date: Aug. 20, 2017

Related U.S. Application Data
(60) Provisional application No. 62/118,053, filed on Feb. 19, 2015.

(51) Int. Cl. G06T 7/00; G06T 7/11; A61B 6/00; A61B 5/08; A61B 5/00; G06T 7/44; G06K 9/46

(52) U.S. Cl. CPC G06T 7/0012; G06T 7/44; G06T 7/11; G06K 9/46; A61B 5/08; A61B 5/7267; A61B 6/50; A61B 6/5217; A61B 6/563; G06T 2207/30061; G06T 2207/10116; G06K 2009/4666; G06K 2209/051

(57) ABSTRACT

A method for estimating a presence of a pneumothorax abnormality. The method comprises classifying at least one texture feature of each of a plurality of pixels of a chest radiograph (CXR) image to generate an output map, identifying at least one lung contour in said CXR image, identifying a plurality of multiple pixel segments along said at least one lung contour, combining values of pixels in each one of said plurality of multiple pixel segments from said output map to generate a global descriptor for said CXR image, and estimating a presence of said pneumothorax abnormality in said CXR image by applying a statistical classifier on said global descriptor.

### BACKGROUND

The present invention, in some embodiments thereof, relates to pneumothorax abnormality detection and, more specifically, but not exclusively, to pneumothorax abnormality detection using image processing techniques.

Pneumothorax is an abnormal accumulation of air in the pleural space that separates the lung from the chest wall. Because of its subtle characteristics, the detection of this abnormality is considered a difficult task among other abnormalities encountered in chest radiograph (CXR) images. Furthermore the extent and location of the abnormality varies greatly between the cases. Examples of pneumothorax abnormality are shown in FIGS. 1A-G which are Frontal upright chest radiographs. FIG. 1A is a radiograph imaging a Normal state chest and FIGS. 1B-1C, 1D-1E, and 1F-1G are pairs of radiographs, the first member of each pair images an abnormality and the second is a zoomed portion of the first member that images the abnormality (e.g. The air accumulation regions are marked by the lines).

### SUMMARY

According to some embodiments of the present invention, there is provided a method for estimating a presence of a pneumothorax abnormality. The method comprises classifying at least one texture feature of each of a plurality of pixels of a chest radiograph (CXR) image to generate an output map, identifying at least one lung contour in the CXR image, identifying a plurality of multiple pixel segments along the at least one lung contour, combining values of pixels in each one of the plurality of multiple pixel segments from the output map to generate a global descriptor for the CXR image, and estimating a presence of the pneumothorax abnormality in the CXR image by applying a statistical classifier on the global descriptor.

Optionally, the classifying comprises calculating at least one value of the at least one texture feature for each one of the plurality of pixels, calculating a plurality of feature descriptors each for another of the at least some pixels and based on respective the at least one value, and compiling the output map mapping each one of the plurality of feature descriptors according to a location of a respective pixel of the plurality of pixels in the CXR image.

Optionally, the classifying comprises applying another statistical classifier on the at least one value to determine a respective the feature descriptor.

More optionally, the another statistical classifier is a Gentle AdaBoost classifier.

Optionally, the at least one texture feature is calculated using local binary patterns (LBP).

Optionally, the at least one texture feature is calculated using Maximum Response 8 (MR8) filter bank.

Optionally, the output map is a binary map.

Optionally, the at least one lung contour comprises a chest outer contour of lungs depicted in the CXR image.

Optionally, the plurality of multiple pixel segments are constant length straight lines originated from a pixel on the at least one lung contour.

Optionally, the statistical classifier is a K-Nearest-Neighbors (KNN) classifier.

Optionally, the at least one texture feature defines a relevancy of a set of pixels around the pixel for identification of a pneumothorax abnormality.

According to some embodiments of the present invention, there is provided a system for estimating a presence of a pneumothorax abnormality. The system comprises an interface adapted to receive a chest radiograph (CXR) image, a memory adapted to store a statistical classifier, a processing unit adapted to: classify each of a plurality of pixels of the CXR image to generate an output map classifying relevancy of a plurality of image parts in the CXR image for identification of a pneumothorax abnormality, identify at least one lung contour in the CXR image, identify a plurality of multiple pixel segments along the at least one lung contour, combine values of pixels in each one of the plurality of multiple pixel segments from the output map to generate a global descriptor for the CXR image, and estimate a presence of the pneumothorax abnormality in the CXR image by applying a statistical classifier on the global descriptor.

According to some embodiments of the present invention, there is provided a method for generating a classifier for estimating a presence of a pneumothorax abnormality. The method comprises aggregating a plurality of values of a plurality of pixels from a plurality of a chest radiograph (CXR) images, at least some of the plurality of CXR images having at least one region marked as a pneumothorax abnormality, calculating a local texture classifier classifying a pneumothorax abnormality texture in a pixel based on an analysis of the plurality of values of the plurality of pixels from the plurality of a chest radiograph (CXR) images, and calculating a global classifier for classifying a global descriptor of a new CXR image based on a training set comprising at least some of the plurality of CXR images and a diagnosis of a presence or an absence of a pneumothorax abnormality. The global descriptor is generated by mapping a plurality of outcomes of applying the local texture classifier on each of a plurality of pixels.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1G are Frontal upright chest radiographs;

FIG. 2 is a flowchart of a method for detection or estimation of a pneumothorax abnormality in a CXR image, according to some embodiments of the present invention;

FIG. 3 is a system for executing classifier for detection or estimation of a pneumothorax abnormality in a CXR image, for instance by implementing the process depicted in FIG. 1, according to some embodiments of the present invention;

FIGS. 4A-4E are Frontal upright chest radiographs having line marking lung contours and upper lung points, according to some embodiments of the present invention;

FIGS. 4F-4G are pairs of images, the first shows how a local abnormality analysis of a normal chest creates an output map and the second shows how a local abnormality analysis of an abnormal chest creates another output map, according to some embodiments of the present invention; and

FIGS. 5A and 5B are an illustration of chest surrounding contour on an image with local analysis values which are aggregated along the lines crossing the contour and computed descriptor values of an image imaging an abnormal chest, according to some embodiments of the present invention; and

FIG. 6 is a flowchart of a method for generating a classifier for estimating a presence of a pneumothorax abnormality, for instance the classifier used as described above, according to some embodiments if the present invention;

FIGS. 7A and 7B are graphs depicting AUC as function of system parameters where the Patch size is M in FIG. 7A and the Global descriptor size is N in FIG. 7B;

FIGS. 7C and 7D are ROC curves for detection of right and left pneumothorax, respectively; and

FIG. 8 is a graph depicting ROC curves for pneumothorax detection, comparison is done by abnormality size.

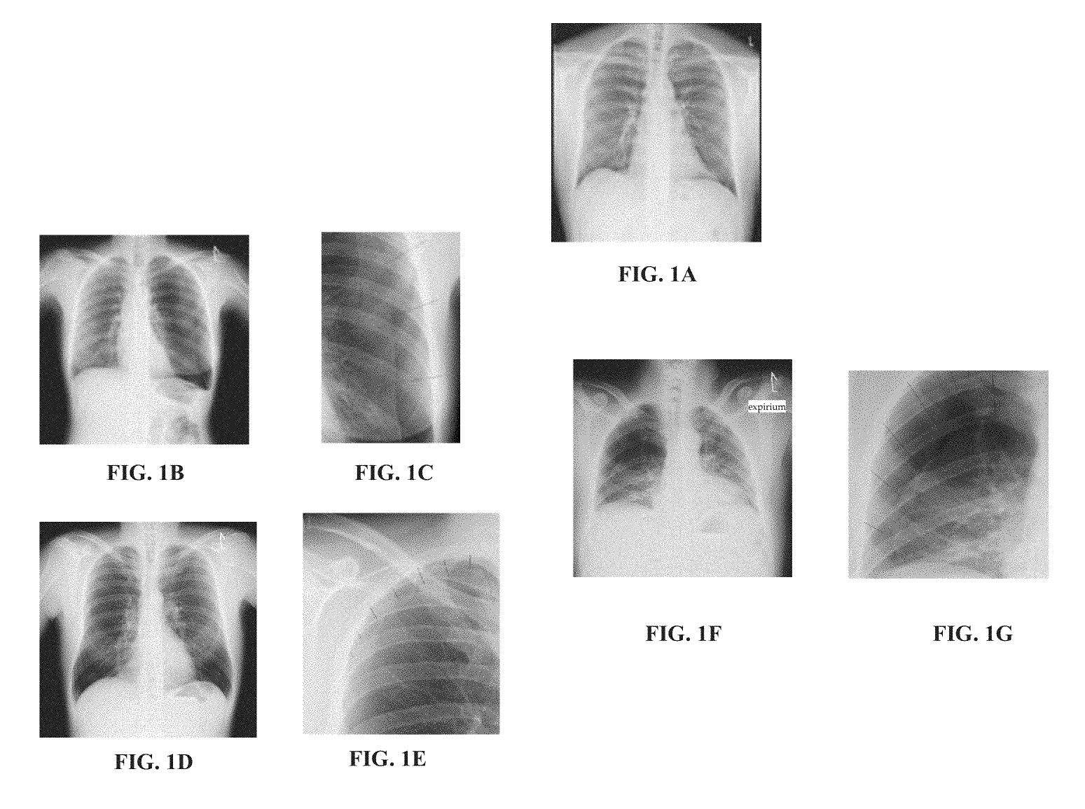

### DETAILED DESCRIPTION

The present invention, in some embodiments thereof, relates to pneumothorax abnormality detection and, more specifically, but not exclusively, to pneumothorax abnormality detection using image processing techniques.

In some embodiments of the present invention, there are provided methods and systems for an automatic detection of pneumothorax abnormality in a CXR image based on local analysis, such as a texture analysis, of a plurality of multiple pixel segments in the CXR image, followed by a unique global representation method. Using the proposed representation, supervised learning is performed in order to determine abnormality detection.

Some embodiments of the present invention are based on advanced image-processing tools and involve automatic tissue characterization, segmentation tools and learning tools. Also, a novel representation and global measure for pathology identification is described.

The methods and systems allow providing a radiologist or any other physician with an automatic estimation of a presence or an absence of a pneumothorax abnormality in a CXR image. This may be used for automatic classification, ranking, and/or urgency prioritization of CXR images.

It should be noted that although the description herein focuses on pneumothorax abnormality detection and estimation, the same processes and methods may be used for detection and estimation of air pockets in the abdominal cavity.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

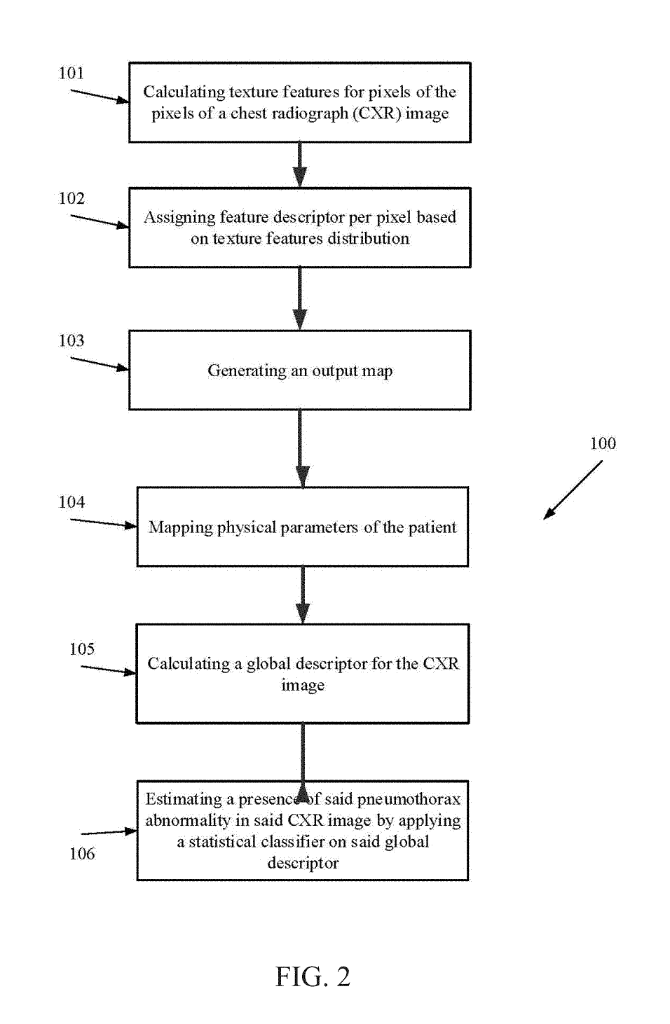

Reference is now made to FIG. 2, which is a flowchart of a method 100 for detection or estimation of a pneumothorax abnormality in a CXR image, according to some embodiments of the present invention. The method is based on localized analysis process, such as a localized texture analysis process, is performed for detection of local abnormalities in multiple pixel segments in the CXR image. Then, a novel global image representation is created and used for detection of the pneumothorax abnormality at the image level. The global image representation may also be used for training a statistical classifier. Optionally, the texture analysis is a local texture analysis which is set to detect a local texture descriptor of the pneumothorax abnormality based on the unique characteristics thereof. A local neighborhood is calculated per pixel in lung portion(s) imaged in the CXR image to allow generating a map discriminating between normal and abnormal regions which suffer from air accumulation inside the lungs. Texture represents characteristics of the pneumothorax abnormality. The local neighborhood around each pixel in the lung may be analyzed to discriminate between normal and abnormal regions inside the lung fields.

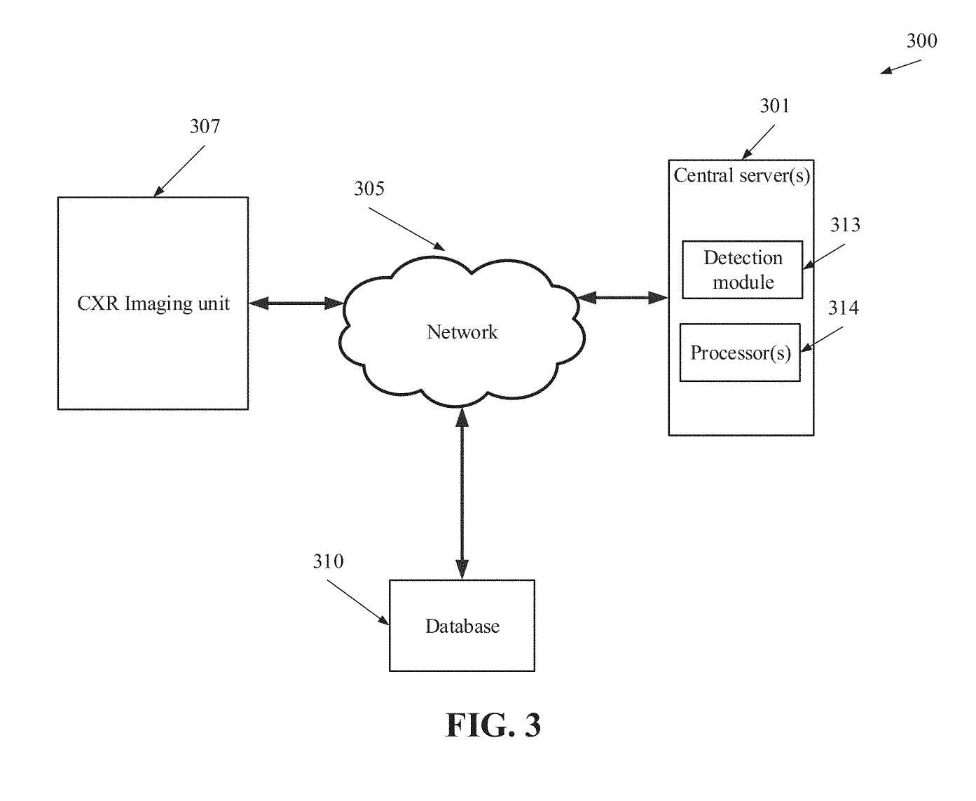

Reference is also made to FIG. 3, which is a system 200 for executing classifier for detection or estimation of a pneumothorax abnormality in a CXR image, for instance by implementing the process depicted in FIG. 2, according to some embodiments of the present invention. The system 200 includes processor(s) for executing a code, referred to herein as a detection module 313, implementing a classifier for performing the localized texture analysis process for detection or estimation of a pneumothorax abnormality in a CXR image, for instance a CXR image captured using a CXR imaging unit 307. The CXR image may be received directly from the CXR imaging unit 307 over a computer network 305 and/or extracted from a database 310 such as an Electronic medical record (EMR) database.

First, as shown at 101, value(s) of one or more texture feature(s) are calculated by executing the detection module 313 for each of some or all of the pixels in the CXR image. For example, local binary patterns (LBP) is calculated, see for example Trefiý, Jirí, and Jirí Matas.“Extended set of local binary patterns for rapid object detection” Proceedings of the Computer Vision Winter Workshop. Vol. 2010. 2010, which is incorporated herein by reference. Additionally or alternatively, rotationally invariant uniform LBP values are calculated, for instance with 4 different radius values. Additionally or alternatively, Maximum Response 8 (MR8) filter bank is used such that for each pixel eight filter responses are obtained from the responses of 38 filters, see for example Ojala, T., Pietikainen, M., & Maenpaa, T. (2002). Multiresolution gray-scale and rotation invariant texture classification with local binary patterns. Pattern Analysis and Machine Intelligence, IEEE Transactions on, 24(7), 971-987 and Varma, M., & Zisserman, A. (2005). A statistical approach to texture classification from single images. International Journal of Computer Vision, 62(1-2), 61-81, which are incorporated herein by reference.

The response vector is optionally quantized to the nearest Texton (dictionary word) using a pre-built dictionary.

Now, as shown at 102, each of these pixels is assigned with a feature descriptor, based on the distribution of the values of the one or more local texture features in a M×M surrounding square that defines the local neighborhood, also referred to as a patch. For example, using the LBP or the MR8 described above, a feature descriptor is assigned to each pixel as the distribution (histogram) of feature values in its M×M square neighborhood (patch). The computation of the local descriptors is done by utilizing the overlap between the surrounding patches of adjacent pixels. Using local histogram of an adjacent neighbor, each local descriptor may be set by updating the histogram with the feature values of the non-overlapping pixels.

The feature descriptors and the CXR image are optionally used for generating and/or updating a local classifier set to classify a pixel based on its feature descriptor.

Optionally, each feature descriptor includes the coordinates of the respective pixel, for example absolute coordinates and/or relative coordinates describing distance from one or more visual objects in the image, for example from the contour defined herein below. When a CXR image used for generating and/or updating a local classifier, such as a pixel level classifier, normal and abnormal regions are manually marked by an operator such as a radiologist, for instance using a designated user interface. A CXR image with marked pixels in the normal and/or abnormal regions is used as a training entry. Each marked pixel constitutes a training set record. Each pixel is represented by a feature descriptor as described above. For example, AdaBoost classifier, such as a Gentle AdaBoost classifier is trained using this training set, see for example Schapire, Robert; Singer, Yoram (1999). “Improved Boosting Algorithms Using Confidence-rated Predictions”. CiteSeerX: 10.1.1.33.4002 and Freund; Schapire (1999). “A Short Introduction to Boosting”.

As shown at 103, the CXR image is processed by classifying each pixel and generating an output map, which are incorporated herein by reference. The above generates a local value map, optionally a binary map, of information from the CXR image, part of which may be irrelevant for identification of pneumothorax abnormality or a gray level map mapping confidence or probability coefficient of a presence or an absence of pneumothorax abnormality in the respective location.

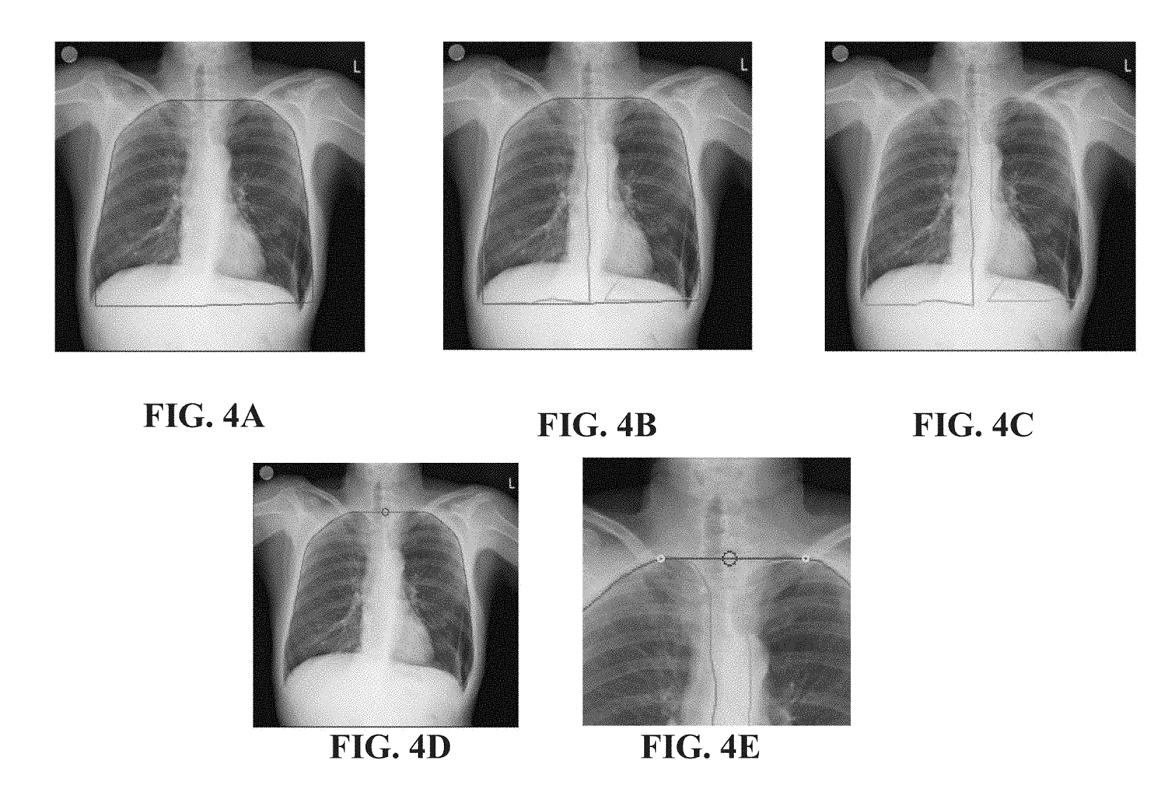

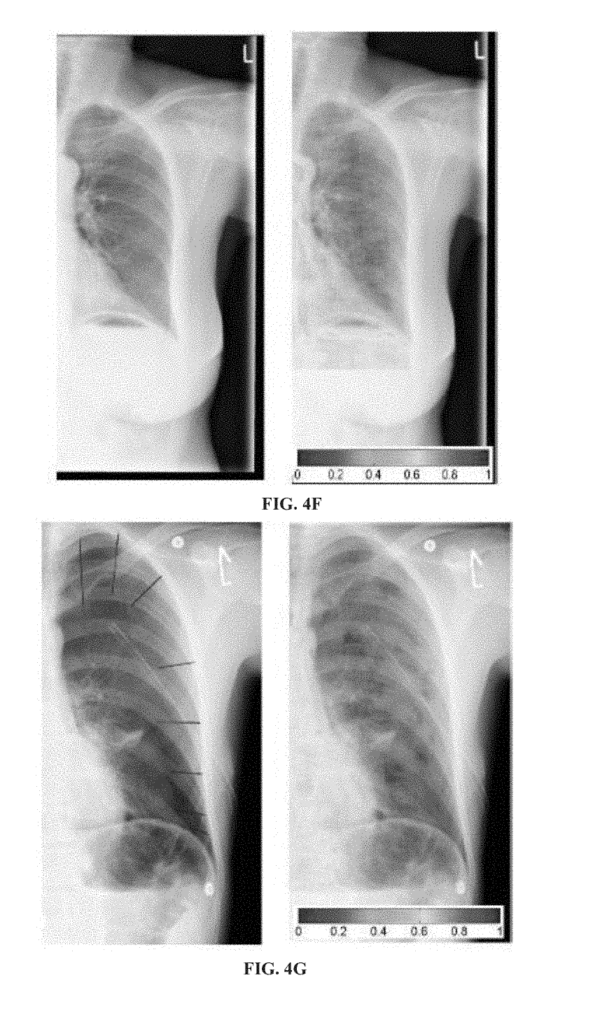

For example, FIGS. 4F and 4G are pairs of images, the first shows how a local abnormality analysis of a normal chest creates an output map and the second shows how a local abnormality analysis of an abnormal chest creates another output map. The air accumulation regions are marked by blue lines. The map values correspond to the estimated probability of abnormality in each pixel. The map included information from the entire radiograph, part of which is irrelevant for identification of pneumothorax. The map may be used for detecting specific spatial distribution of values characteristic of the pneumothorax pathology as described below.

As shown at 104, after the local texture analysis is completed, a map, optionally adjustable to the physical parameters of the patient, of estimating spatial spread of the pneumothorax abnormality is used for applying global detection of pneumothorax abnormality in the CXR image. For example, as indicated below a contour of lungs is set and used for selecting multi pixel segments used in the global detection process.

Optionally, a chest wall contour detection procedure is applied. The process may consist of segmenting two lung fields using a method3 based on Active Contour algorithm (Kass et al. (1988)). Then a surrounding contour Clungs may be created. The surrounding contour points are set as the convex hull vertices of the union of both segments points. The points of the Clungs contour may be checked sequentially, until two consecutive points, each of which originated in a different lung segment. Next, the chest top point may be chosen as the mid-point between the two detected points. The mid-upper part of the full contour may be selected by moving (along the Clungs contour points) a constant distance, D from the top point in both directions. The distance D can be determined in several ways to preserve robustness to the size variations between subjects. In this framework, the D value may be set to be about 30% of the length of the Clungs contour. This yielded a mid-upper contour, chest wall, having a length of about 60% of the length of the Clungs contour.

As shown at 105, the local analysis output, the above local value map, is incorporated into a global detection decision by calculating a global image descriptor for the CXR image. The global image descriptor may be calculated and optionally trained as follows:

First, an organ visual pattern, such as a lung contour is calculated. For instance, a chest outer contour is calculated based on the external boundaries of both lungs fields.

Optionally, a chest outer contour is constructed as follows:

First, each lung is segmented, for example using a segmentation tool which is based on an Active Contour method for segmentation.

Then, a contour that surrounds both lung segments is calculated, for example using convex-hull vertices of the union of both segments points, see also the lungs segmentation output (both total lungs and left and right lungs) in FIGS. 4A-4C.

Now, a localization of the top point is calculated by moving along the surrounding contour points, until two consecutive points, each one of them originated in different lung segment, are detected. This allows choosing the top point to the mid-point between the two detected points.

A partial contour may be constructed by selecting the mid-upper part of the full contour by moving, along the contour points, from the top point in both directions a constant distance, denoted herein as D. D may be determined in several ways in order to preserve robustness to the size variations between examined subjects. In the suggested framework, D value is set to be 30% of the length of the fully surrounding contour. This yields a mid-upper contour whose length is 60% of the length of the whole contour. For example, FIG. 4D depicts final partial (mid-upper) contour and FIG. 4E depicts two lateral points that represent two consecutive vertices in the convex-hull point series, which each one of them belongs to different lung segment. A top point location is set to be the mid-point between them.

Optionally, resampling of each contour coordinate series is performed, leading to a representation by a constant number of points, denoted herein by N.

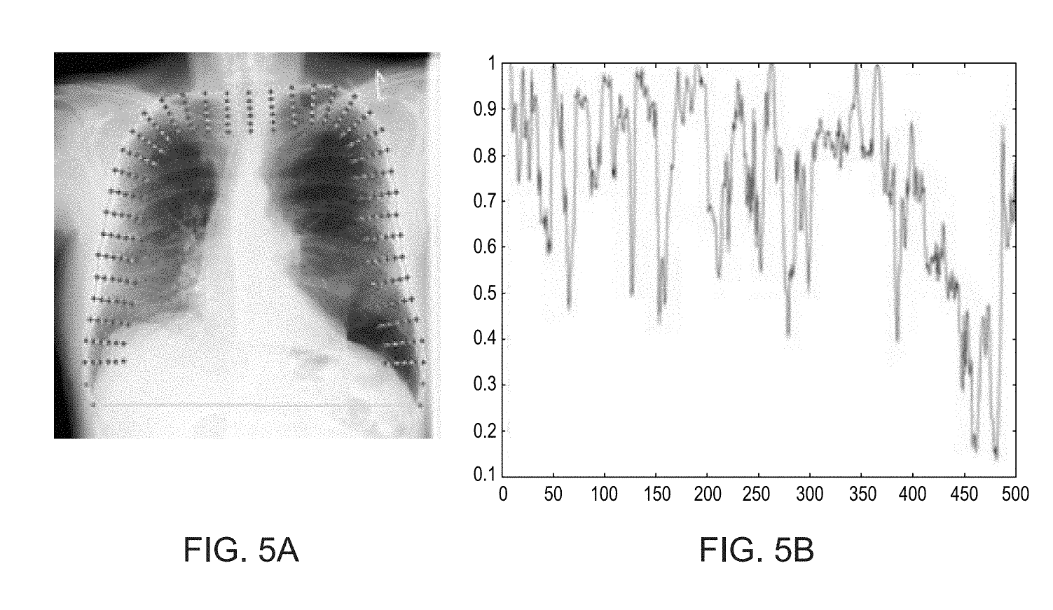

Each point on the constructed lung visual pattern is assigned with a multiple pixel segment, such as a straight line, optionally with a constant length or an adaptive length that is determined based on size of organ(s) in the CXR image and/or physiological parameters of the patient. The origin of each constant length straight line lies in its corresponding contour point, and its direction is towards the inner lung field, with direction chosen to be a normal vector to the lung visual pattern, see for example FIG. 5A is an illustration of chest surrounding contour on an image and local analysis values which are aggregated along the lines crossing the contour. See also FIG. 5B which is an example of computed descriptor values of an image imaging an abnormal chest where the horizontal axis denotes a number of points along the contour and the vertical axis denotes a proportion of abnormal pixels along each line.

Now, along each multiple pixel segment, such as a constant length straight line, corresponding values of the local value map, for instance the binary map, obtained from the local analysis are accumulated (e.g. summed or averaged). Each contour point is assigned with its corresponding line accumulated value, leading to a representation by a N-dimensional descriptor. For example, FIG. 5A depicts chest surrounding contour determined by convex-hull vertices of set of points which belong to the lungs segments contours. The surrounding contours are marked in the image by lines. This global descriptor for the given CXR image is based on aggregation of relevant local descriptors, such as descriptors which are based on texture analysis.

Using measurements along the constructed chest outer contour, each CXR is represented by an N dimensional descriptor. Additionally or alternatively, a pooling step in the representation process may be performed. For each CXR, the descriptor values are combined, for instance summed to create a graph as depicted in FIG. 5B, along the N/2 coordinates in one lung side (e.g. relative to the top point as central lung marker) and compared versus the sum of N/2 coordinates in another lung (e.g. relative to the top point as central lung marker). The coordinate set with the lowest sum is then discarded, yielding an N/2 dimensional descriptor.

Now, as shown at 106, the constructed global descriptors may be used for a supervised learning process on the given dataset, with each training CXR image labeled as normal/abnormal by a radiologist. Classification is performed using a statistical classifier, such as a K-Nearest-Neighbors (KNN) classifier or a support vector machine (SVM) classifier.

The global image descriptor may be used for supervised classification to categorize the image as either normal or pathological. The image first undergoes the texture analysis process to produce the local abnormality maps as described above and the global descriptor that utilizes the chest wall contour is generated. This descriptor is used to produce a decision label for the tested image.

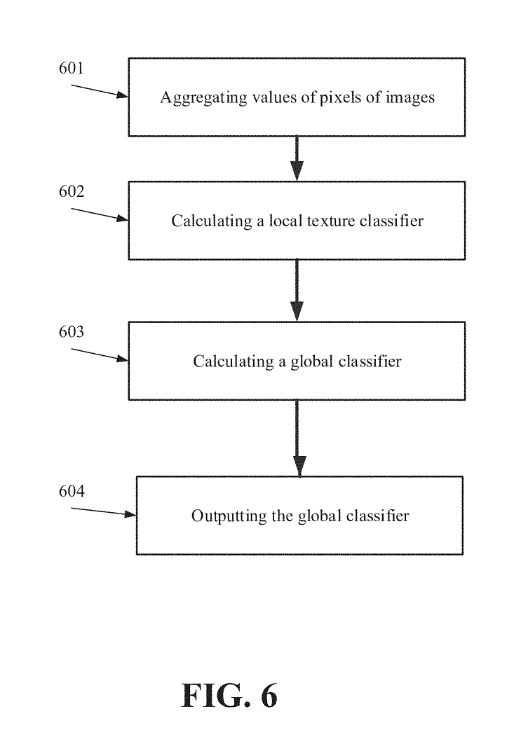

Reference is also made to FIG. 6 which is a flowchart of a method for generating a classifier for estimating a presence of a pneumothorax abnormality, for instance the classifier used as described above, according to some embodiments if the present invention. First, as shown at 601, a plurality of values of a plurality of pixels are aggregated from a plurality of CXR images where at least some of the CXR images having region(s) marked as a pneumothorax abnormality. As shown at 602, a local texture classifier classifying a pneumothorax abnormality texture in a pixel may now by calculated based on an analysis of the plurality of values. The calculation may be done by executing a designated code by the processor(s) 314 of the system 301. Now, as shown at 603, a global classifier may be calculated, for instance by the processor(s) 314 of the system 301, for classifying a global descriptor of a new CXR image based on a training set comprising at least some of the CXR images and a diagnosis of a presence or an absence of a pneumothorax abnormality. The global descriptor is generated by mapping a plurality of outcomes of the applying of the local texture classifier on each of the pixels of each of the images. As shown at 604 the global classifier is outputted for being used as described above.

The above process allows using texture features for analysis of local areas inside the lung fields, in order to detect abnormal texture caused by the air accumulation. This approach is not based on line finding methods in order to detect the boundary of the pneumothorax abnormality pattern but rather on a global descriptor which captures the unique pneumothorax properties that appear in many typical pneumothorax abnormalities.

The methods as described above are used in the fabrication of integrated circuit chips.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant methods and systems will be developed and the scope of the term a CXR image, a processor, and a system is intended to include all such new technologies a priori.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Various embodiments and aspects of the present invention as delineated hereinabove and as claimed in the claims section below find experimental support in the following examples.

### Examples

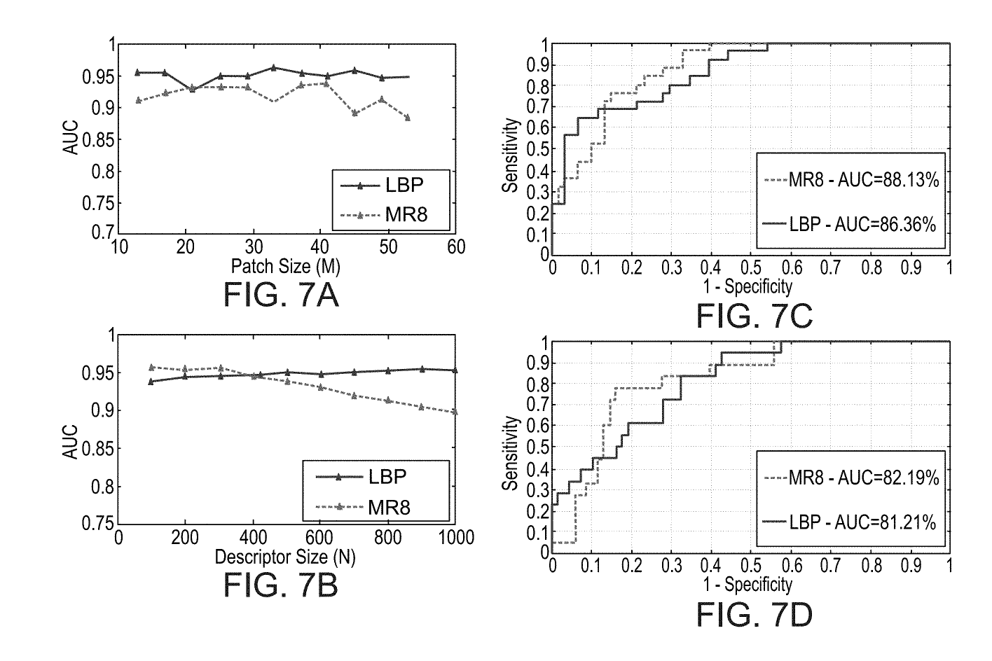

Reference is now made to the following examples, which together with the above descriptions, illustrate some embodiments of the invention in a non limiting fashion. In these examples a dataset consisted of frontal (PA) upright CXRs obtained at Sheba Medical Center is used. The dataset is divided into two sets, A and B. Using the CXRs of dataset A, the training process of both local and global model was performed. To examine the robustness of the system with respect to its parameters, detection results were collected for varying parameter values. In the following experiments, dataset A was divided randomly into training and validation sets for the evaluation. Tests were performed for detection at the patient level, without localization of the abnormality using the method described above). In the first experiment we examined the effect of the local patch size (M) used in the local analysis stage. As seen in FIG. 7A (AUC as function of system parameters), detection ability was stable and the characteristic local abnormalities could be captured even in relatively small neighborhood. The influence of the global descriptor size (N), which corresponds to the number of sampled points on the chest wall contour, was examined. The results showed in FIG. 7B a negligible effect on performance. Further to the model development and tuning of parameters, an evaluation of the performance of the proposed framework was carried out. Both local and global model were trained with the labeled CXRs of dataset A. The models were trained using a local patch size of 41×41 pixels and with a global descriptor size of length N=500. Using the trained model, the system was tested on the CXRs of dataset B. The 95% confidence intervals of the obtained area under curve (AUC) were calculated using a bootstrap sampling method (Efron (1979)), computing statistics with 10,000 bootstrap samples. FIG. 7C and FIG. 7D and Table 1 show the calculated ROC curves for the pathology detection performance and the obtained area under curve (AUC) values (the two figures correspond to detection results for the two sides of the chest):

TABLE 1

| AUC | Right Pneumothorax | Left Pneumothorax |
|---|---|---|
| LBP | 0.86 [0.76-0.93] | 0.81 [0.69-0.9] |
| MR8 | 0.88 [0.8-0.94] | 0.82 [0.69-0.9] |

The best performance was observed with MR8 as local feature set and yielded an AUC of 0.88 and 0.82 for the right and left pneumothorax respectively. Sensitivity and specificity values are also displayed in Table 2 based on the optimal cut-off point—the ROC point closest to (0,1); see Table 2:

| | | Right Pneumothorax | Left Pneumothorax |
|---|---|---|---|
| LBP | SEN | 0.68 | 0.83 |
| | SPEC | 0.89 | 0.68 |
| MR8 | SEN | 0.84 | 0.78 |
| | SPEC | 0.77 | 0.84 |

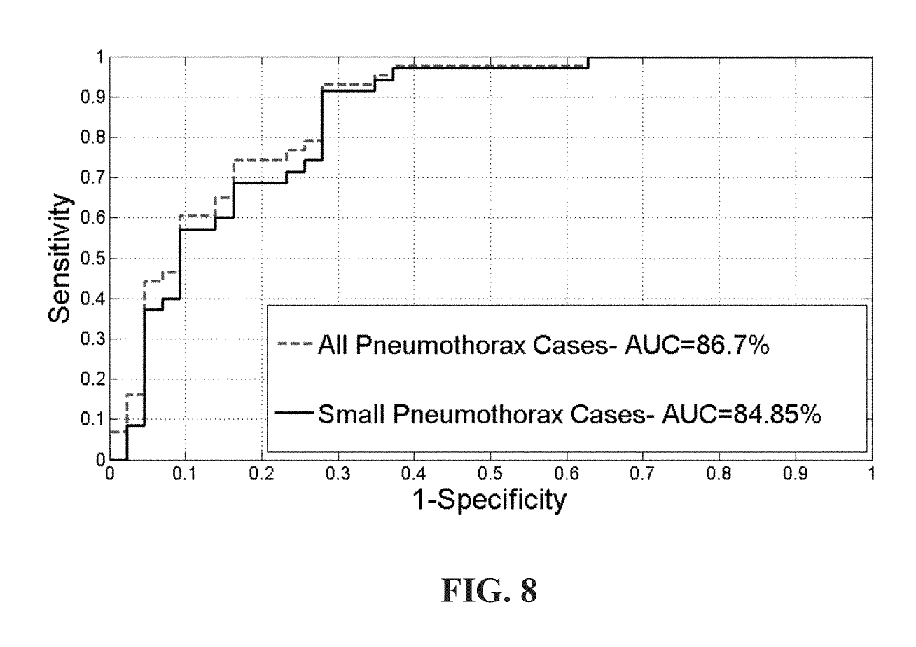

An additional experiment was carried out to assess detection performance included pneumothorax cases that had been categorized as ‘small’. To compare to the general case, detection tests were performed at the patient level, using MR8 as local feature set. FIG. 8 displays the comparison between the obtained ROC curve when testing only for ‘small’ pneumothorax and the computed curve in the general case. The obtained area under curve values are displayed in Table 3:

| | Small Pneumothorax | All Pneumothorax Cases |
|---|---|---|
| AUC | 0.85 [0.74-0.92] | 0.87 [0.77-0.93] |

The influence of the supervised learning method at the global level, by comparing detection performance of the SVM classifier against the Random Forest (RF) classifier (Breiman (2001)) is also investigated. In the comparison the MR8 is used as the local feature set. 3,000 trees are used for the random forest configuration. As can be seen in the results in Table 4, SVM performed slightly better in detection of the left pneumothorax, whereas the RF achieved higher AUC values in detection of the right pneumothorax; see Table 4:

| AUC | Right Pneumothorax | Left Pneumothorax |
|---|---|---|
| SVM | 0.88 [0.8-0.94] | 0.82 [0.69-0.9] |
| RF | 0.91 [0.82-0.96] | 0.8 [0.68-0.88] |

The testing set in this experiment only It is expected that during the life of a patent maturing from this application many relevant methods and systems will be developed and the scope of the term a network, a client, a device and a processor is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method for estimating a presence of a pneumothorax abnormality, comprising:
classifying at least one texture feature of each of a plurality of pixels of a chest radiograph (CXR) image to generate an output map;
identifying at least one lung contour in said CXR image;
identifying a plurality of multiple pixel segments along said at least one lung contour;
combining values of pixels in each one of said plurality of multiple pixel segments from said output map to generate a global descriptor for said CXR image; and
estimating a presence of said pneumothorax abnormality in said CXR image by applying a statistical classifier on said global descriptor.

2. The method of claim 1, wherein said classifying comprises:
calculating at least one value of said at least one texture feature for each one of said plurality of pixels;
calculating a plurality of feature descriptors each for another of said at least some pixels and based on respective said at least one value;
compiling said output map mapping each one of said plurality of feature descriptors according to a location of a respective pixel of said plurality of pixels in said CXR image.

3. The method of claim 2, wherein said classifying comprises applying another statistical classifier on said at least one value to determine a respective said feature descriptor.

4. The method of claim 3, wherein said another statistical classifier is a Gentle AdaBoost classifier.

5. The method of claim 1, wherein said at least one texture feature is calculated using local binary patterns (LBP).

6. The method of claim 1, wherein said at least one texture feature is calculated using Maximum Response 8 (MR8) filter bank.

7. The method of claim 1, wherein said output map is a binary map.

8. The method of claim 1, wherein said at least one lung contour comprises a chest outer contour of lungs depicted in said CXR image.

9. The method of claim 1, wherein said plurality of multiple pixel segments are constant length straight lines originated from a pixel on said at least one lung contour.

10. The method of claim 1, wherein said statistical classifier is a K-Nearest-Neighbors (KNN) classifier.

11. The method of claim 1, wherein said at least one texture feature defines a relevancy of a set of pixels around said pixel for identification of a pneumothorax abnormality.

12. A system for estimating a presence of a pneumothorax abnormality, comprising:
an interface adapted to receive a chest radiograph (CXR) image;
a memory adapted to store a statistical classifier;
a processing unit adapted to:
classify each of a plurality of pixels of said CXR image to generate an output map classifying relevancy of a plurality of image parts in said CXR image for identification of a pneumothorax abnormality;
identify at least one lung contour in said CXR image;
identify a plurality of multiple pixel segments along said at least one lung contour;
combine values of pixels in each one of said plurality of multiple pixel segments from said output map to generate a global descriptor for said CXR image; and
estimate a presence of said pneumothorax abnormality in said CXR image by applying a statistical classifier on said global descriptor.

13. A method for generating a classifier for estimating a presence of a pneumothorax abnormality, comprising:
aggregating a plurality of values of a plurality of pixels from a plurality of a chest radiograph (CXR) images, at least some of said plurality of CXR images having at least one region marked as a pneumothorax abnormality;
calculating a local texture classifier classifying a pneumothorax abnormality texture in a pixel based on an analysis of said plurality of values of said plurality of pixels from said plurality of a chest radiograph (CXR) images;
calculating a global classifier for classifying a global descriptor of a new CXR image based on a training set comprising at least some of said plurality of CXR images and a diagnosis of a presence or an absence of a pneumothorax abnormality;
wherein global descriptor is generated by mapping a plurality of outcomes of applying said local texture classifier on each of a plurality of pixels; and
outputting said global classifier.
